(19) United States
(12) Patent Application Publication
(10) Pub. No.: US 2025/0122553 A1
(43) Pub. Date: Apr. 17, 2025

(54) PLATFORM FOR ANTIMICROBIAL SUSCEPTIBILITY TESTING AND METHODS OF USE THEREOF

(71) Applicant: ASTRADX, INC., Chestnut Hill, MA (US)

(72) Inventor: Sarmad Muneeb Siddiqui, Fremont, CA (US)

(21) Appl. No.: 18/688,593

(22) PCT Filed: Sep. 2, 2022
(86) PCT No.: PCT/US2022/042509

§ 371 Date: Mar. 1, 2024

Related U.S. Application Data
(60) Provisional application No. 63/240,653, filed on Sep. 3, 2021.

(51) Int. Cl. C12Q 1/20; C12M 1/32; C12M 1/34; G06T 5/70; G06T 7/00

(52) U.S. Cl. CPC C12Q 1/20; C12M 23/12; C12M 41/46; G06T 5/70; G06T 7/0012; G06T 2207/30024; G06T 2207/30072

(57) ABSTRACT

Systems and methods for quickly determining antibacterial or antimicrobial susceptibility using an innovative growth dynamic model are disclosed. A system includes an image collection subsystem constructed and arranged to generate a plurality of images of a microbial sample. The system also includes an image analysis subsystem comprising a non-transitory computer-readable medium storing thereon sequences of computer-executable instructions for determining the susceptibility of the microbial species from the image of the microbial sample that, when executed by one or more processors, cause the one or more processors to perform operations that calculate the susceptibility of the microbial species by determining one or both of microbial species replication and microbial species stasis in the presence of the antimicrobial agent from the manipulation of the changing pixel intensities. A non-transitory computer-readable medium storing thereon instructions for determining antibacterial or antimicrobial susceptibility are also disclosed.

### SUMMARY

In accordance with one aspect, there is provided a system for determining a susceptibility of a microbial species in the presence of an antimicrobial agent. The system may include an image collection subsystem constructed and arranged to generate a plurality of images of a microbial sample. The system further may include an image analysis subsystem including a non-transitory computer-readable medium storing thereon sequences of computer-executable instructions for determining the susceptibility of the microbial species from the plurality of images of the microbial sample. When executed by one or more processors, the sequences of computer-executable instructions stored on the non-transitory computer-readable medium cause the one or more processors to perform operations including i) receiving, from the image collection subsystem, one or more of the plurality of images of the microbial sample; ii) extracting data corresponding to a pixel intensity of one or more regions of the one or more of the plurality of images; iii) reducing intensity variations in the per pixel intensity of the one or more regions of the one or more of the plurality of images; and iv) calculating the susceptibility of the microbial species by determining one or both of microbial species replication and microbial species stasis in the presence of the antimicrobial agent from the manipulation of the changing pixel intensities.

In some embodiments, the image collection subsystem may include a light source, a photosensitive element constructed and arranged to collect light from the light source that has transmitted through the microbial sample, and a memory for storing the plurality of images representative of the collected transmitted light from the microbial sample.

In some embodiments, determining the susceptibility of the microbial species may include one or more of: a) reducing noise in the pixel intensity of one or more regions of the one or more of the plurality of images; b) removing statistical outliers from the pixel intensity of the one or more regions of the one or more of the plurality of images; and/or c) fitting the pixel intensity of the one or more regions of the one or more of the plurality of images to a model representative of a growth dynamic of the microbial species to determine the susceptibility.

In further embodiments, the image analysis subsystem may be configured to display the results of the image analysis to a user. For example, the displayed results may be used to determine a treatment course for a patient. In particular embodiments, the displayed results may be used for epidemiological purposes, e.g., determining which antibiotic or antimicrobials to keep in stock for clinical use.

In some embodiments, the microbial species may include at least one species from the genus Acinetobacter, Escherichia, Klebsiella, Pseudomonas, Enterococcus, Streptococcus, and Staphylococcus. In certain embodiments, the microbial species may be selected from A. baumannii, E. coli, K. pneumoniae, P. aeruginosa, and S. aureus. The system disclosed herein is not limited to the analysis of growth of these exemplary genera or species.

In some embodiments, the microbial species may be grown for less than or about 12 hours during collection of the plurality of images. In some embodiments, the microbial species may be grown for less than or about 9 hours during collection of the plurality of images. the microbial species may be grown for less than or about 6 hours during collection of the plurality of images. For example, the microbial species may be grown for less than or about three 3 hours, e.g., less than about 3 hours, less than about 2.5 hours, less than about 2 hours, less than about 1.5 hours, or less than about 1 hour during image acquisition.

In specific embodiments, the microbial sample includes a well plate having a plurality of wells each separated by at least one surrounding interwell region, the microbial sample including microbial growth in a portion of the plurality of wells. In certain embodiments, the one or more regions of the at least one of the plurality of images correspond to the plurality of wells and the associated at least one surrounding interwell region.

In some embodiments, reducing intensity variations may include correcting the pixel intensity of the pixels in each of the plurality of wells using the pixel intensities of the associated at least one surrounding interwell region. In some embodiments, reducing noise may include performing independent component analysis (ICA) on the variation reduced pixel intensity data of the pixels in each of the plurality of wells to generate at least one signal corresponding to microbial growth and at least one signal corresponding to growth inhibition from the antimicrobial agent. In some embodiments, removing statistical outliers may include performing one or both of a mean absolute deviation calculation and a k-means clustering calculation on the noise reduced pixel intensity data.

In some embodiments, wherein fitting the pixel intensity comprises fitting the outlier reduced pixel intensity data to a growth dynamic model comprising one or more phenomenological models. In particular embodiments, the growth dynamic model comprises a combined multi-dimensional growth dynamic model comprising the Gompertz model and the Hill model.

In further embodiments, the image analysis subsystem may be configured to calculate the minimum inhibitory concentration (MIC) of the antimicrobial agent.

In accordance with an aspect, there is provided a method of determining a susceptibility of a microbial species in the presence of an antimicrobial agent. The method may include acquiring a plurality of images of a microbial sample using an image collection system. The method may include sending or transmitting one or more of the plurality of images to an image analysis system comprising a non-transitory computer-readable medium storing thereon sequences of computer-executable instructions for determining the susceptibility of the microbial species from the plurality of images of the microbial sample by manipulating data corresponding to a pixel intensity of one or more regions of one or more of the plurality of images to a hybrid model representative of a growth dynamic of the microbial species. The method further may include calculating a minimum inhibitory concentration (MIC) of the antimicrobial agent from the one or more of the plurality of images by determining one or both of microbial species replication and microbial species stasis in the presence of the antimicrobial agent from the determined growth dynamic. The method additionally may include storing or providing the calculated MIC to a user.

In further embodiments, determining the susceptibility of the microbial species from the plurality of images of the microbial sample may include extracting data corresponding to a pixel intensity of one or more regions of the one or more of the plurality of images. In further embodiments, determining the susceptibility of the microbial species from the plurality of images of the microbial sample may include reducing intensity variations in the pixel intensity of the one or more regions of the one or more of the plurality of images. In further embodiments, determining the susceptibility of the microbial species from the plurality of images of the microbial sample may include reducing noise in the pixel intensity of one or more regions of the one or more of the plurality of images. In further embodiments, determining the susceptibility of the microbial species from the plurality of images of the microbial sample may include removing statistical outliers from the pixel intensity of the one or more regions of the one or more of the plurality of images.

In some embodiments, the hybrid model comprises a combined multi-dimensional growth dynamic model comprising the Gompertz model and the Hill model.

In accordance with an aspect, there is provided a non-transitory computer-readable medium storing instruction which, when executed by a computer, cause the computer to perform a method. The method may include acquiring a plurality of images of a microbial sample using an image collection system. The method further may include determining from analysis of one or more of the plurality of images of the microbial sample a growth dynamic including one or both of microbial species replication and microbial species stasis in the presence of an antimicrobial agent. The method additionally may include calculating a minimum inhibitory concentration (MIC) of the antimicrobial agent from the determined microbial growth dynamic in the one or more of the plurality of images of the microbial sample.

In certain embodiments, the step of determining the growth dynamic comprises determining the growth dynamic using a combined multi-dimensional growth dynamic model comprising the Gompertz model and the Hill model.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates a schematic of a system for determining a susceptibility of a microbial species in the presence of an antimicrobial agent, according to an embodiment of this disclosure;

FIG. 2 illustrates a portion of a 384 well microwell plate simulation with landmarks defined;

FIG. 3 illustrates a 384 well microwell plate where pixel intensities of each well are equalized;

FIG. 4 illustrates an equalized interwell signal over time using a model according to an embodiment of this disclosure;

FIGS. 5A and 5B illustrate independent component analysis (ICA) modified signals. FIG. 5A illustrates ICA-decomposed latent sources of the imaging data using two components. FIG. 5B illustrates the reconstruction of the de-noised imaging data based on the two latent sources with the solid lines showing the original data;

FIGS. 6A-6F illustrate replicate filtering and statistical analysis of the filtered data. FIGS. 6A and 6B illustrate the median (FIG. 6A) and mean (FIG. 6B) of replicate intensity versus time for select wells from a 384 well microplate. FIGS. 6C and 6D illustrate the corresponding mean absolute deviation (MAD) value for the data in FIG. 6A and FIG. 6B, respectively. FIG. 6E illustrates a comparison in the original unfiltered mean (blue) and mean of the filtered data after k-means clustering (black). FIG. 6F illustrates the MAD value for the data in FIG. 6E;

FIGS. 7A-7B illustrate a comparison of the fit to a prior growth model using a prior image analysis scheme (FIG. 7A) and the image analysis scheme according to an embodiment of this disclosure (FIG. 7B);

FIGS. 8A-8B illustrate a comparison of the fit to a prior growth model using a prior image analysis scheme (FIG. 8A) and an image analysis scheme according to an embodiment of this disclosure (FIG. 8B);

FIG. 9 illustrates a comparison between the modeled growth of a prior growth model (blue line), the Gompertz model (yellow line), and a model according to an embodiment of this disclosure (red line); and

FIGS. 10A-10D illustrate 3D surface plots of processed image data (FIGS. 10A and 10C) and the model fit according to an embodiment of this disclosure (FIGS. 10B and 10D) as a function of time and log (concentration).

### DETAILED DESCRIPTION

The invention relates to the fields of cell growth and detection. In many industries, particularly the food, beverage, healthcare, electronic, livestock/animal husbandry, biotechnology, and pharmaceutical industries, it is essential to rapidly analyze samples for the degree of contamination by microorganisms, such as bacteria, yeasts, or molds.

Conventional methods used in clinical laboratories worldwide require isolation of bacteria on culture plates as single bacterial colonies. The colonies are then used to set up one of several culturing methods, e.g., the broth microdilution reference method, agar dilution, disk diffusion, gradient diffusion, or several commercial methods that are either modified versions of the broth microdilution method or extrapolate the results from the broth microdilution method based on growth kinetics of organisms in culture. Available testing is limited to first-line drugs. In practice, these methods provide antimicrobial susceptibility testing (AST) results in a minimum of two days after specimen receipt in the clinical lab. This testing generally requires at least one day to isolate pure bacterial colonies, and one additional day to obtain the AST results from these colonies. With emerging antimicrobial resistance, this two-or-more day delay may lead to adverse clinical outcomes.

Systems that provide faster AST results from pure bacterial colonies are notably expensive and thus cost prohibitive. Some AST instruments can cost over $100,000 and still can take seven or more hours to run an AST analysis after isolating a pure bacterial colony. The cost of testing a single sample can be in excess of $200. As a result of their high cost, rapid AST systems would not likely be widely available throughout the world, including many parts of the United States, in rural areas, and in developing countries.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

“About” and “approximately” shall generally mean an acceptable degree of error for the quantity measured given the nature or precision of the measurements. Exemplary degrees of error are within 20 percent (%), typically within 15%, more typically within 10%, and even more typically, within 5% of a given value or range of values.

As used interchangeably herein, the terms “subject,” “patient,” and “individual” refer to any organism to which a therapeutic agent in accordance with the invention may be administered, e.g., for experimental, diagnostic, prophylactic, and/or therapeutic purposes. Typical subjects include any animal (e.g., mammals, such as mice, rats, cats, dogs, pigs, horses, rabbits, non-human primates, and humans). A subject may seek or be in need of treatment, require treatment, be receiving treatment, be receiving treatment in the future, or be a human or animal who is under care by a trained professional for a particular disease or condition.

“Microbial species” as used herein, encompasses lifeforms including bacteria, e.g., mycobacteria, enterobacteria, non-fermenting bacteria, and Gram-positive or Gram-negative cocci or rods, archaea, fungi. i.e., yeasts and molds, algae, protozoa, and viruses.

“Microbial sample” as used herein, can refer to any suitable apparatus capable of holding a microbial sample such that the sample can be grown for analysis and imaged. For example, a microbial sample may include a Petri dish, a well plate, e.g., a 6, 12, 24, 48, 96, 384 or 1536 well microplate, a microscope slide, a glass plate with isolated droplets, or any other suitable holder for a microbial species.

“Images” as used herein, can refer to still photographic images collected using any suitable apparatus configured for such purpose or a video collected over a period of time using any suitable apparatus configured for such purpose. Still images may be isolated from a video accordingly.

“Light source” as used herein, refer to any suitable source of electromagnetic radiation for collecting an image of a microbial sample. Example sources can include visible light, infrared light, and ultraviolet light. Light from the light source may be of any suitable plane polarization, e.g., p-polarization or s-polarization, or unpolarized, i.e., random direction, light.

“Pixel intensity” as used herein refers to the brightness and/or color of an identified pixel. Low brightness is considered to have low to zero intensity and high brightness is high intensity. Darker colors are considered to have low to zero intensity and brighter colors are considered to have high intensity.

In accordance with an aspect, there is provided a system for the determination of a susceptibility of a microbial species in the presence of an antimicrobial agent. The system may comprise an image collection subsystem constructed and arranged to generate a plurality of images of a microbial sample. The system further may comprise an image analysis subsystem comprising a non-transitory computer-readable medium storing thereon sequences of computer-executable instructions for determining the susceptibility of the microbial species from the plurality of images of the microbial sample. When executed by the computer, the sequences of computer-executable instructions stored on non-transitory computer-readable medium causes the computer to perform operations including, for example receiving from the image collection subsystem one or more of the plurality of images of the microbial sample. The operations performed by the computer when executing the instructions stored on the non-transitory computer-readable medium further may include extracting data corresponding to a pixel intensity of one or more regions of the one or more of the plurality of images. The one or more regions of the at least one of the plurality of images may correspond to the plurality of wells and the associated at least one surrounding interwell region surrounding each of the plurality of wells. The operations performed by the computer when executing the instructions stored on the non-transitory computer-readable medium additionally may include reducing intensity variations in the per pixel intensity of the one or more regions of the one or more of the plurality of images. The operations performed by the computer when executing the instructions stored on the non-transitory computer-readable medium additionally may include calculating the susceptibility of the microbial species by determining one or both of microbial species replication and microbial species stasis in the presence of the antimicrobial agent from the manipulation of the changing pixel intensities.

Systems and methods of this disclosure provide for a rapid determination of microbial species growth and the determination of a minimum inhibitory concentration (MIC) of an antibiotic or antimicrobial compound for a microbial species. This rapid determination of the MIC associated with a particular species provides benefits for treatment determination in a clinical setting. For example, when a patient in a clinical setting is suspected of having an infection, they are generally given a pharmaceutical, such as an antibiotic, that represents the best guess as to what will effectively treat the infecting pathogen. Generally, for bacterial infections, broad-spectrum antibiotics are given with the hope of covering any potential pathogen. Once the AST results are available, directed therapy, tailored to the susceptibility profile of the pathogen, can be given. This treatment path fails to account for microbial life that has developed resistance to pharmaceutical treatments, and further fails to account for the overall impacts of broad-spectrum antibiotics or antimicrobial compounds on beneficial microbial life. Having specific information on the MIC and any observed resistance to pharmaceutical treatments such as provided by the systems and methods of this disclosure can allow for the precise tailoring of treatment regimens on a timescale that prevents additional delay and any adverse outcomes associated with said delays.

Information, i.e., MIC associated with specific microbial species, may also be used for epidemiological purposes. For example, in a clinical setting, e.g., a hospital, when microorganisms are isolated from two individual patients have the same pattern in their MIC in response to the administration of an antibiotic or other antimicrobial, this raises the suspicion that the isolated microorganisms originated from the same source. Further, hospitals and public-health laboratories routinely produce summary statistics for each microbial species encountered frequently among its patients. In so doing, these institutions generally record how often the MIC was, e.g., 0.5, vs. 1, vs. 2, vs. 4, and so on, producing histograms that track changes in MIC. Following these histograms over time may be useful for quantifying the spread of antibiotic or antimicrobial resistance, which may provide useful guidance for aiding in the procurement of one or more proper antibiotics or other antimicrobial agents to keep on hand to treat the microorganism, when its presence is observed.

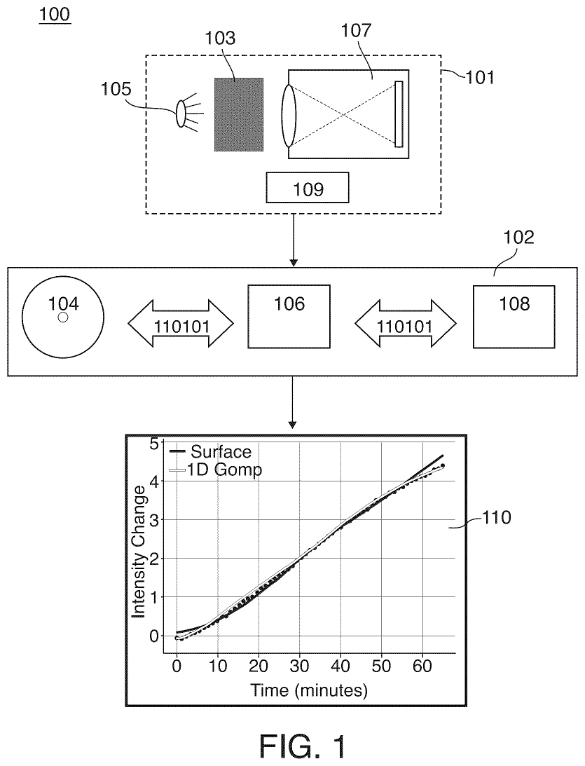

As illustrated in FIG. 1, a system 100 for the determination of a susceptibility of a microbial species in the presence of an antimicrobial agent comprises an image collection subsystem 101, and image analysis subsystem 102, and an output 110 to display the results of the image analysis to a user or operator. The image collection subsystem 101 may include a light source 105, a camera comprising a photosensitive element 107 constructed and arranged to collect light from the light source 105 that has transmitted through a microbial sample 103, and a memory 109 for storing an image representative of the collected transmitted light from the microbial sample 103. The microbial sample 103 may be any suitable sample containing microorganisms, such as a well plate, Petri dish, or the like. Alternatively, the microbial sample 103 may include aerosolized or nebulized droplets of a fluid including the microbial species deposited onto a suitably transparent or translucent substrate, e.g., a glass plate or the like, sufficiently spaced apart to minimize droplet coalescence. An image collection subsystem 101 may be any suitable image collection subsystem having any combination of light sources, photodetection elements, and connections to other system components, and this disclosure is in no way limited to the example image collection subsystem shown and described.

With continued reference to FIG. 1, a system 100 for the determination of a susceptibility of a microbial species in the presence of an antimicrobial agent comprises an image analysis subsystem 102. The image analysis subsystem 102 may include a non-transitory computer-readable medium 104 having computer-executable instructions stored thereon for determining the susceptibility of the microbial species from the plurality of images of the microbial sample. The non-transitory computer-readable medium 104 may include, for example, a disk, e.g., a hard disk drive (HDD), solid state drive (SSD), or flash memory. Typically, in operation, the CPU of computer 108 causes data to be read from the non-transitory computer-readable medium 104 into another memory 106 that allows for faster access to the information by the computer 108 than does the non-transitory computer-readable medium 104. This memory 106 is typically a volatile, random access memory such as DRAM or SRAM as described herein. The computer 108 generally manipulates the data within its internal memory and then copies the data to the non-transitory computer-readable medium 104 after processing is completed. A variety of mechanisms are known for managing data movement between the non-transitory computer-readable medium 104 and the internal memory 106 of the computer 108, and embodiments disclosed herein are not limited to any particular data movement mechanism.

With continued reference to FIG. 1, a system 100 further may comprise a display 110 for the output of the operations performed from the execution of the instructions for determining the susceptibility of the microbial species in the presence of the antimicrobial agent. The display 110 may by any type of display, such as a visual output or a file containing the resultant analysis, or both, and embodiments disclosed herein are in no way limited to any particular data output or data display mechanism.

Systems and methods disclosed herein are generally performed using computers to acquire the plurality of images of the microbial samples, transmit the acquired images, e.g., still photographic images of the microbial sample collected over a timeseries or a video of the microbial sample, to the image analysis subsystem, and produce the desired output, e.g., a MIC or other related output. In some embodiments, bootstrapping a single synthetic data set may be used to generate sufficient data to establish the central tendency, i.e., a central or typical value for a probability distribution such as a mean, median or mode, corresponding to the growth of the microbial species. In some embodiments, for the analysis of images of microbial samples that include motion blur, e.g., blurred still photographs or videos, one or more deconvolution techniques, such as Richardson-Lucy deconvolution or Wiener deconvolution, may be applied. The addition of deconvolution as part of the image analysis subsystem may allow for images to be acquired at a faster rate. i.e., an increased temporal resolution. In further embodiments, artifacts present in the plurality of images of the microbial samples may be removed using one or more classical digital signal processing techniques. As a non-limiting example, high frequency artifacts present in the plurality of images of the microbial samples may be removed by the application of a low-pass filter.

One or more parts of systems and methods disclosed can be achieved by using artificial intelligence for automation, such as unsupervised learning approaches. For example, the artificial intelligence that acquires and analyzes images may include a neural network. Neural networks are patterned mathematically to acquire, process, and interpret incoming information in a manner similar to the human brain, e.g., by taking input information and passing it along to at least one “neuron,” further propagating information until terminating at an output. By passing information along to multiple “neurons” the neural network is able to improve the way in which it interprets an input signal, i.e., it learns from previous input signals, thereby improving the accuracy of the end result. The “neurons” are typically organized in layers. Different layers may perform different kinds of transformations on their inputs. Another non-limiting example of artificial intelligence for one or more of the systems and methods disclosed herein is cluster analysis, where sets of data are iteratively grouped based around one or more specific properties, such as a density or a centroid of a set of values. An exemplary clustering model for use with data that varies in time is k-means clustering, where a mixed set of data can be grouped into k clusters, with k being a natural number, and each data point in the set belonging to the nearest mean. Other types of unsupervised iterative models for analyzing data corresponding to the plurality of images of microbial samples collected by image collection subsystems disclosed herein and the specific types recited herein are in no way limiting.

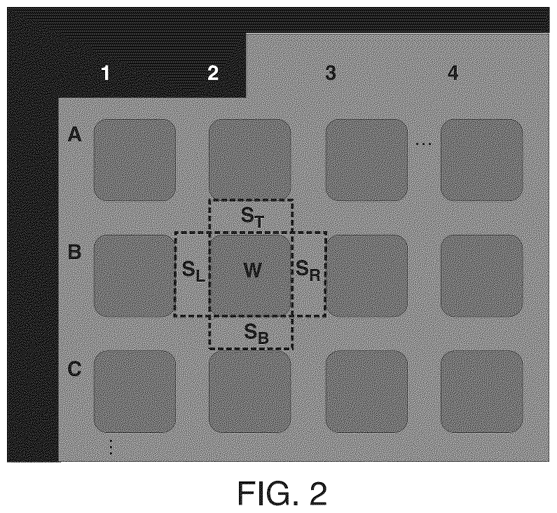

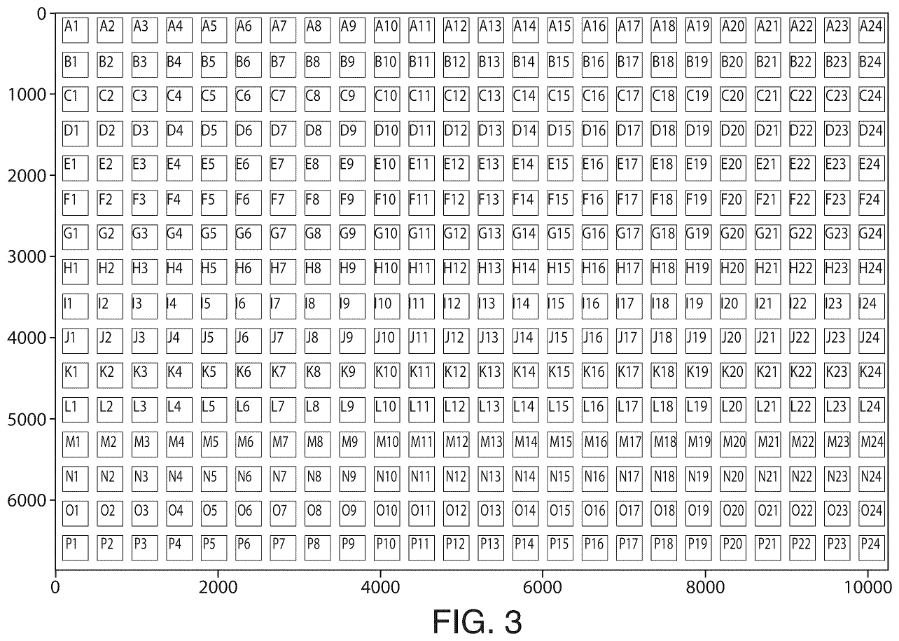

In some embodiments, determining the susceptibility of the microbial species comprises may include a step of reducing intensity variations in the pixel intensity of the one or more regions of the one or more of the plurality of images. This is also known as image equalization, and in some embodiments may include correcting the pixel intensity of the pixels in each of the plurality of wells using the pixel intensities of the associated at least one surrounding interwell region. The general framework underlying the reduction of pixel intensity variations is the a priori expectation that areas of a microbial sample that do not have any microbial activity, e.g., the interwell regions if a 384 well plate, should all have equal mean pixel intensity when imaged minus some variance, such as from manufacturing defects in the material used to manufacture the sample carrier. There are a number of approaches that may be used for reducing intensity variations in the pixel intensity of the one or more regions of the one or more of the plurality of images. In some cases, a two-step image equalization process may be used. A global image equalization may be performed by calculating the global average intensity over all non-sample regions, e.g., the interwell regions in a well plate-based sample, and applying the difference in intensity from the global average as a correction factor. The correction factor, i.e., the delta (A) intensity, can then be used to calculate a region-specific correction, e.g., a per-well correction in a 384 well plate. As a non-limiting example, a global interwell intensity average may be calculated across all timepoints. i.e., in a plurality of images taken in time. This type of correction can then be used to correct the intensity of each individual pixel in an image, with the amount of correction being a function of the distance between a surrounding non-sample containing, e.g., interwell, region and the specific sample area pixel, e.g., well pixel, being corrected. For example, a pixel near the top of one of the plurality of images of a microbial sample (in a well) will be less influenced by a correction factor from an interwell region near the bottom of the same image of the microbial sample. With reference to FIGS. 2 and 3, i.e., a 384 well plate, pixel corrections may be made in one or both of the row-wise dimensions or the column-wise directions. The image equalization schemes described in this disclosure are in no way limited to those described and other available schemes are within the scope of this disclosure.

In some embodiments, determining the susceptibility of the microbial species comprises may include a step of reducing noise in the pixel intensity of one or more regions of the one or more of the plurality of images. As described herein, reducing noise in the pixel intensity of one or more regions of the one or more of the plurality of images may be reducing timepoint-to-timepoint variance of said pixel intensities.

In general, microbial growth can be considered a multicomponent process, including a lag phase before microbial growth begins, a log phase of exponential growth classically represented as a logarithmic function, and a stationary phase once the carrying capacity of the environment is reached. Rather than be bound by the classical approach, this disclosure considers the dynamics of microbial growth as statistically separable and weighted independent biological processes, for example growth/cell division and stasis, without the need to fit to any classical model. The reduction in noise, i.e., timepoint-to-timepoint variance, of said pixel intensities thus can be considered a separation of the growth and stasis processes from each region of a microbial sample, such as each well in a 384 well plate, and determination of the statistical weights for these processes, with noise reduction occurring as it cannot be part of either growth or stasis. There are any number of different approaches for separating a signal of interest from a collection of mixed signals. The separation of signals may be achieved by using independent component analysis (ICA), an unsupervised statistical technique that extracts individual source signals from the measured mixture signal. For example, in some embodiments, reducing noise may include performing ICA on the variation reduced pixel intensity data of the pixels in each of the of the one or more regions of the one or more of the plurality of images to generate at least one signal corresponding to microbial growth and at least one signal corresponding to growth inhibition from the antimicrobial agent. There are other similar techniques that can separate one or more specific signals from a mixed source, including, but not limited to, principal component analysis (PCA), singular value decomposition, dependent component analysis, non-negative matrix factorization, and stationary subspace analysis, among others. In some embodiments, one or more specific approaches for noise reduction may be used. For example, the noise reduction may first utilize a technique for reducing the dimensionality of the source signal, such as PCA, then separation of signals of interest from the reduced dimensionality source signal using a different noise reduction technique, such as ICA. The noise reduction schemes described in this disclosure are in no way limited to those described and other available schemes are within the scope of this disclosure.

In particular embodiments, the dimensionality of the source signal is reduced using PCA, for example, by determining the number of components that explained the substantial majority of any variance in the data. The dimensionality reduced data can be separated into individual components using ICA, of which the two resulting signals can be considered to track the processes of microbial species replication and of the inhibition of microbial species growth, e.g., by an antibiotic or other antimicrobial compound. Without wishing to be bound by any particular theory, it is believed that dimensionality reduction using PCA may be able to remove a portion, e.g., a majority, of the noise from the data. Thus, in some cases, with the majority of the noise removed by reducing dimensionality, application of ICA to separate out signals of interest provide for the original signal per region of the microbial sample, such as the per well signal of a 384 well plate.

In some embodiments, determining the susceptibility of the microbial species may include a step of removing statistical outliers from the pixel intensity of the one or more regions of the one or more of the plurality of images. In a typical microbial growth experiment, there may be particular regions, such as individual wells in a 384 well plate for example, where microbial life fails to grow and produce a detectable result. The inclusion of failed microbial growth in the calculation of a microbial growth rate may artificially lower the predicted growth rate relative to the true growth rate, an important consideration for the determining the MIC of an antibiotic or other antimicrobial compound. The present disclosure contemplates the filtering of the pixel intensities of each of the plurality of images using a statistical filter, such as by calculating a mean and mean absolute deviation (MAD) of the original data. The MAD is the average distance between each set of data points and the mean. The resulting MAD can be evaluated against a threshold value to determine whether a specific replicate should be excluded from the dataset. As used herein, a “replicate” is a well of a well plate with the same contents, i.e., the same organism, and the same antibiotic at the same concentration. The threshold value may be determined experimentally or from previous compiled information on similar microbial growth. Once outliers are removed, replicates in the remaining pixel intensity data from each of the plurality of images can be calculated using one or more filtering steps. As noted herein, calculation steps such as replicate filtering may be performed using artificial intelligence, such as an unsupervised learning process. In this context, the results of replicate filtration can be used to train the image analysis subsystem to improve performance over time. As the goal is to remove replicates, one approach is to use one or more analysis techniques to group similar datapoints together, such as by cluster analysis. There are a number of suitable cluster techniques which may be used for replicate removal including, but not limited to, connectivity clustering, centroid clustering, statistical distribution clustering, and density clustering, among others. An exemplary clustering technique to filter replicate datapoints in each timeseries, i.e., the plurality of images, is k-means clustering as described herein. A k-means algorithm often assigns each point to a cluster for which the center (also referred to as a centroid) is nearest. The center often is the average of all the points in the cluster, that is, its coordinates often are the arithmetic mean for each dimension separately over all the points in the cluster. A number of clusters can be selected as appropriate. An appropriate number of dimensions used in determining clusters can be selected as appropriate. The replicate filtering schemes described in this disclosure are in no way limited to those described and other available schemes are within the scope of this disclosure.

In some embodiments, determining the susceptibility of the microbial species comprises may include a step of fitting the pixel intensity of the one or more regions of the one or more of the plurality of images to a model representative of a growth dynamic of the microbial species to determine the susceptibility. As described herein, microbial species growth is generally modeled on a sigmoidal curve, known as a Gompertz model, representing the lag phase, exponential growth, and a reduction in growth once carrying capacity is reached. In some prior treatments, microbial growth following this type of curve is approximated by the classic “hockey stick” fit based loosely on experimental evidence of bacterial growth in a closed system. Traditional models suffer from overfitting data and ignoring information that can be derived from the changes in growth across varying concentrations of antimicrobial or antibiotic compounds. One approach to improve the prediction of microbial species growth is to extend a classical model to be hybrid growth dynamic that, in addition to considering growth at given antimicrobial or antibiotic concentrations, also considers the dose response of microbial growth to an antimicrobial or antibiotic at a given time. An exemplary concentration-based model for microbial growth is called the Hill model, which is a modified logistic function whose inflection point corresponds to the minimum inhibitory concentration (MIC) of the antimicrobial or antibiotic. The hybrid growth dynamic model incorporating both the concentration dependence and time dependence of exposure to antimicrobial or antibiotic compounds on microbial species growth provides for an improved model for determining the MIC compared to classical approaches for modeling microbial species growth while being less susceptible to overfitting and non-biological dependencies in modeled growth processes.

As described herein, traditional AST in clinical and non-clinical settings is generally a slow process, requiring hours to days in order to culture and observe sufficient colony formation to enable appropriate determinations on susceptibility and other epidemiological considerations. Using an image analysis subsystem as described herein allows for the rapid determination of microbial species growth that is on a scale of a factor of two or more lower than that of traditional AST methods. In general, images of the microbial sample are acquired about once per minute during the growth of the microbial sample. In some embodiments, the images of the microbial sample are acquired about once per every 45 seconds, about once per every 40 seconds, about once per every 35 seconds, about once per every 30 seconds, about once per every 25 seconds, about once per every 20 seconds, about once per every 15 seconds, about once per every 10 seconds, about once per every 9 seconds, about once per every 8 seconds, about once per every 7 seconds, about once per every 6 seconds, about once per every 5 seconds, about once per every 4 seconds, about once per every 3 seconds, about once per every 2 seconds, or about once per every second. In some embodiments, the microbial species may be grown during image acquisition for less than or about 12 hours, e.g., less than about 12 hours, less than about 11.5 hours, less than about 11 hours, less than about 10.5 hours, less than about 10 hours, less than about 9.5 hours, less than about 9 hours, less than about 8.5 hours, less than about 8 hours, less than about 7.5 hours, less than about 7 hours, less than about 6.5 hours, less than about 6 hours, less than about 5.5 hours, less than about 5 hours, less than about 4.5 hours, less than about 4 hours, less than about 3.5 hours, less than about 3 hours, less than about 2.5 hours, less than about 2 hours, less than about 1.5 hours, or less than about 1 hour during image acquisition. Under these conditions, detectable changes in microbial species growth may be observed in less than about 10 minutes, and statistical confidence in the detection and quantification of microbial species growth may be achieved in less than about 30 minutes. The rapidity of which the disclosed systems and methods can detect and model microbial species growth provides for a determination of the time and concentration dependence on said microbial growth even in the absence of more refined modeling, additional signal inputs, or further experimental steps. Further, as one or more analysis techniques incorporated into the image analysis subsystem may include artificial intelligence components, such as unsupervised learning, e.g., neural networks, clustering algorithms, and the like, the resulting growth dynamic and MIC determinations generally will increase in accuracy and precision the more image analysis that occurs, thus decreasing the duration necessary to determine microbial species growth and MIC in a specific sample.

In some embodiments, the microbial species may include one or more species including bacteria, e.g., mycobacteria, enterobacteria, non-fermenting bacteria, and Gram-positive or Gram-negative cocci or rods, archaea, fungi, i.e., yeasts and molds, algae, protozoa, and viruses. For example, the microbial species may include at least one species from the genus Acinetobacter, Enterococcus, Escherichia, Klebsiella, Pseudomonas, and Staphylococcus. In specific embodiments, the microbial species is selected from A. baumannii, E. coli, K. pneumoniae, P. aeruginosa, and S. aureus. The recited microbial species are exemplary, and this disclosure is in no way limited by the specific microbial species under study and analysis.

In accordance with an aspect, there is provided a method of determining a susceptibility of a microbial species in the presence of an antimicrobial agent. The method may comprise acquiring a plurality of images of a microbial sample using an image collection system. The method may comprise sending or transmitting one or more of the plurality of images to an image analysis system comprising a non-transitory computer-readable medium storing thereon sequences of computer-executable instructions for determining the susceptibility of the microbial species from the image of the microbial sample. The non-transitory computer-readable medium storing thereon sequences of computer-executable instructions may include instruction for manipulating data corresponding to a pixel intensity of one or more regions of one or more of the plurality of images to a hybrid model representative of a growth dynamic of the microbial species. The method further may comprise calculating a minimum inhibitory concentration (MIC) of the antimicrobial agent from the one or more of the plurality of images by determining one or both of microbial species replication and microbial species stasis in the presence of the antimicrobial agent from the determined growth dynamic. The method additionally may comprise storing or providing the calculated MIC to a user.

In some embodiments, determining the susceptibility of the microbial species further comprises extracting data corresponding to a pixel intensity of one or more regions of the one or more of the plurality of images. In some embodiments, determining the susceptibility of the microbial species further comprises reducing intensity variations in the pixel intensity of the one or more regions of the one or more of the plurality of images. In some embodiments, determining the susceptibility of the microbial species further comprises reducing random noise in the pixel intensity of one or more regions of the one or more of the plurality of images. In some embodiments, determining the susceptibility of the microbial species further comprises removing statistical outliers from the pixel intensity of the one or more regions of the one or more of the plurality of images. In particular embodiments, the hybrid model used to determine the susceptibility of the microbial species comprises a combined multi-dimensional growth dynamic model comprising the Gompertz model and the Hill model.

In accordance with an aspect, there is provided a non-transitory computer-readable medium having a computer-readable algorithm stored thereon that defines instructions that, as a result of being executed by a computer, causes the computer to perform a method determining a susceptibility of a microbial species in the presence of an antimicrobial agent. The method to be performed upon execution of the instructions stored on the non-transitory computer-readable medium may include acquiring a plurality of images of a microbial sample using an image collection system. The method to be performed further may include determining from analysis of one or more of the plurality of images of the microbial sample a growth dynamic in the presence of an antimicrobial agent. The method to be performed additionally may include calculating a minimum inhibitory concentration (MIC) of the antimicrobial agent from the determined microbial growth dynamic in the one or more of the plurality of images of the microbial sample.

In some embodiments, the step of determining the growth dynamic comprises determining the growth dynamic using a hybrid multi-dimensional growth dynamic model comprising the Gompertz model and the Hill model.

### Examples

The function and advantages of these and other embodiments can be better understood from the following examples. These examples are intended to be illustrative in nature and are not considered to be in any way limiting the scope of the invention.

In the following Example, it is demonstrated that the image analysis as described herein provides for more rapid detection of microbial growth in the presence of antimicrobial agents compared to existing model. As described herein, the microbial sample may include a standard laboratory microwell plate, such as a 384 well plate illustrated in FIGS. 2 and 3. The image analysis method described in this Example is not solely limited to the analysis of a 384 well plate, and any sample holder, such as a smaller or larger well plate, a Petri dish, a microscope slide, or a glass plate, or the like may be imaged. A first step in performing the image analysis is in defining the well and interwell regions of a standard microwell plate.

As illustrated in FIG. 2, in a 384-well plate used as a microbial sample, each well is labeled using an alphanumeric key, with rows labeled A-P and columns labeled 1-24. An arbitrary well is referred to as W, and specific wells are notated by their position on the grid of well, i.e., the upper leftmost well is W_{A1}. The interwell regions, i.e., the area surrounding the four sides a well, is referred to as S_{rc} (for “surrounding”). Each S_{rc} is subscripted by its location around a specific W. For example, as illustrated in FIG. 2, each W has a S_{T}, S_{B}, S_{L}, and S_{R}, for top, bottom, left, and right, respectively. In the present disclosure, a baseline image correction is performed on each well and interwell region using the following procedure:
1. A mask for the interwell region (I_{S}) is generated. Thresholding or a similar edge/object detection algorithm is used to generate the masked image.
2. The masked image is used to generate a global interwell average (S_{g}) across all timepoints. This is a scalar quantity that we assume is the true mean intensity of all the interwell regions on the plate. The interwell region is replaced with this value.
3. A delta interwell image is generated: I_{Δ}=Iₛ−S_{g}
4. The mean delta for the S_{subregion} of each S_{rc} for every timepoint is calculated along its larger matrix dimension:

$${Δ}_{T} = mean ( {S}_{T} , axis = rows )$$

$${Δ}_{R} = mean ( {S}_{R} , axis = columns )$$

$${Δ}_{B} = mean ( {S}_{B} , axis = rows )$$

$${Δ}_{L} = mean ( {S}_{L} , axis = columns )$$

Each pixel (p) in each well W is then corrected according to:

$${p}_{rc}^{′} = {p}_{rc} - 0.5 * [ {Δ}_{T} * ( 1 - \frac{r}{m} ) + {Δ}_{B} * ( \frac{r}{m} ) + {Δ}_{L} * ( 1 - \frac{c}{n} ) + {Δ}_{R} * ( \frac{c}{n} ) ]$$

where dim (W)=(m,n).

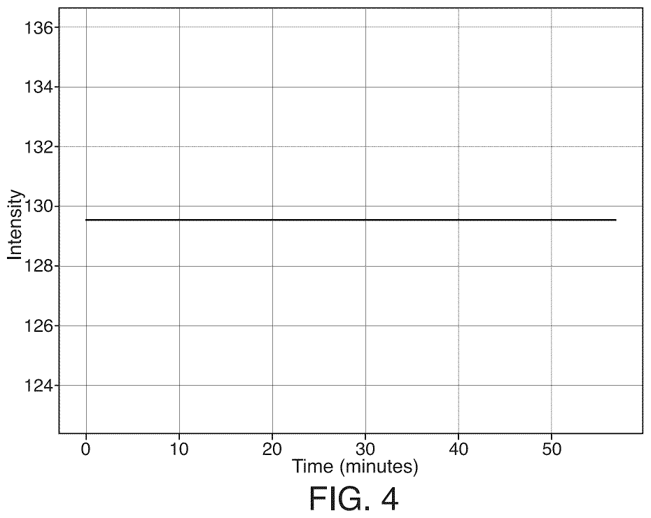

This equation states that the true value of a pixel p′ in a given well W can be estimated as subtracting the Δₓ from the pixel p. However, the influence of a given sub-interwell region will be a function of the distance from the pixel, i.e., a pixel zero rows below Δ_{T} will have a maximum influence from that correction whereas a pixel at the bottom row of W will have minimal influence from Δ_{T}. This relationship is inverted for corrections stemming from Δ_{B}. The equation also has a weight of 0.5 to account for correction being made in both row-wise and column-wise directions. The results of the correction algorithm can be seen in FIGS. 3 and 4.

The next step in processing an image of a well plate is minimizing timepoint-to-timepoint variance using independent component analysis. As described herein, microbial growth has often been modeled as a generalized logistic function with three phases: a log phase of exponential growth, and a stationary phase once the carrying capacity of the environment is reached. In this disclosure, microbial growth is not assigned to a specific function; rather, the dynamics of growth as noted herein are seen as statistically separable independent biological processes including growth/cell division and stasis. This separation of independent biological processes from the extracted pixel data is performed using Independent Component Analysis (ICA). As it pertains to the pixel intensities in each well from an image of a microwell plate, the mixed intensity signals from each well can be represented mathematically as:

$$\begin{matrix}{x}_{1} ( t ) = {a}_{11} {s}_{1} ( t ) + {a}_{12} {s}_{2} ( t ) … + {a}_{1 n} {s}_{n} ( t ) \\ {x}_{2} ( t ) = {a}_{21} {s}_{1} ( t ) + {a}_{22} {s}_{2} ( t ) … + {a}_{2 n} {s}_{n} ( t ) \\ ⋮ \\ {x}_{m} ( t ) = {a}_{m 1} {s}_{1} ( t ) + {a}_{m 2} {s}_{2} ( t ) … + {a}_{m n} {s}_{n} ( t )\end{matrix}$$

where xₘ(t) is the mixed signals observed, sₙ(t) are latent source signals, and a₁₁ . . . aₙₘ are unknown coefficients that when linearly combined with the latent sources, lead to the mixed signal. The above system of equations is often re-written in matrix form:

$$X = A S$$

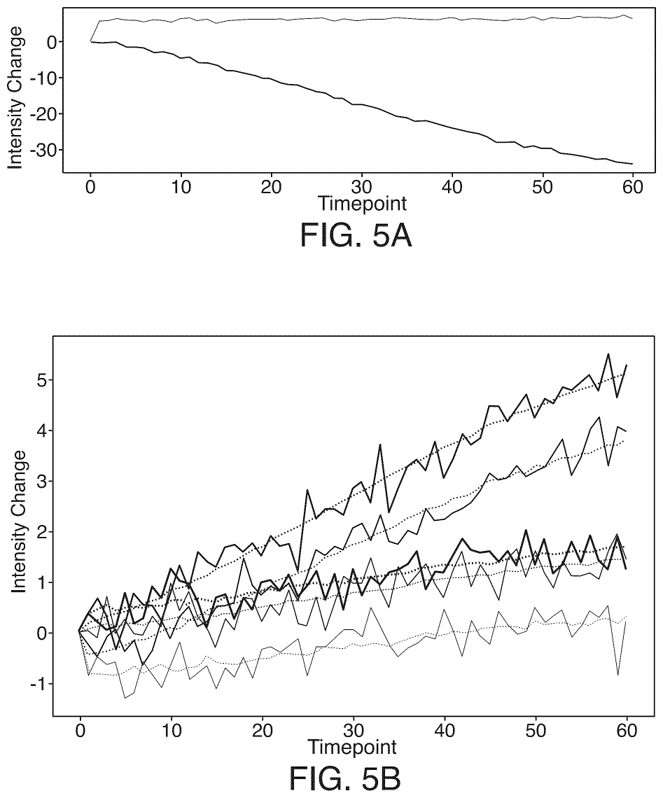

If the coefficients of A were known this would be a linear system of equations that could be solved conventionally. However, the goal is to estimate both the unknown S and A. ICA enables estimation of both S and A if the signals are statistically independent and non-Gaussian in nature. Here, the noisy image data yield the observed time series, i.e., one per well (X), S is the latent source signal comprising the dynamical processes, and A is the “mixing matrix” that holds the weights, which when multiplied by the latent sources generates the time series, modulo noise. x₁(t) . . . xₘ(t) are the well (m=384) intensities as a function of time (typically t≤75). S is of size n*t where n is the number of latent signal sources that is solved for (here, 2). The maximum number of sources is m. This systematic process further includes the option to use principal component analysis (PCA) to reduce the dimensions of signal sources and then apply ICA to separate each signal source into independent components. After multiple trials with dimensionality reduction, it was found that n=2 components explained >90% of the variance in the data, and once ICA was applied, the two source signals could be interpreted as the processes of bacterial replication and of inhibition of bacterial growth (e.g., by an antibiotic or other antimicrobial). Gaussian or random noise was removed because it cannot be a part of either process, per the framework of ICA. The results of an ICA decomposition of mixed-signal input data into two separate components is illustrated in FIGS. 5A and 5B, where FIG. 5A illustrates ICA for one mixed signal and FIG. 5B illustrates a comparison between ICA fitted and experimental data for five individual wells on a well plate.

The next step in processing an image of a well plate is handling growth failures, which are to be expected in any microbial culture. As noted herein, prior models often incorporate those wells which fail to show any observable growth, resulting in a lower predicted growth rate compared to the true growth rate. Here, a two-step procedure is performed on the original set of replicate data to exclude such extreme outliers to generate a more physiologically relevant estimate of replicates' central tendency.

The first step uses the mean absolute deviation (MAD) statistic to filter the original set of replicates. The mean and the MAD of the replicate timeseries are calculated as follows:

$$\overline{T} = \frac{1}{n} {{\sum }_{i = 1}}^{n} {T}_{i}$$

$$MAD = \frac{1}{n} {\sum }_{i = 1}^{n} \vert {T}_{i} - \overline{T} \vert$$

If for a given timeseries Tᵢ the ratio of the absolute deviation to the MAD is greater than a defined threshold (experimentally determined), it is excluded from the dataset:

$${T}_{i} ∉ T , if \frac{\vert {T}_{i} - \overline{T} \vert}{M A D} > 2.4$$

The second step was further filtering the filtered set of replicates via k-means clustering. The k-means algorithm minimizes the sum of the within-cluster sum-of-squares using the expectation-maximization (EM) algorithm:

$$min {\sum }_{i = 1}^{k} {\sum }_{x ∈ {S}_{i}} {\vert x - {μ}_{i} \vert}^{2}$$

where x={x₁, x₂, x₃ . . . } is a set of observations, S=k clusters, and μᵢ is the mean of points in cluster Si. The filtered n timeseries are partitioned into either two or three clusters, where one of the clusters must have at most two timeseries. This assumption is based on the principle that if the clusters contain a similar number of timeseries the data variance is high, and not that there are one or two outliers in the filtered data. The MAD of each cluster is calculated. If the ratio of the MAD of a cluster to the MAD of the filtered T is less than 0.8, or the Silhouette score is >0.6, the sub-cluster is selected. The Silhouette score is defined as:

$$s = \frac{b - a}{max ( a , b )}$$

where a is the mean distance between a sampled point and all the points in the same cluster, and b is the mean distance between a sampled point and all the points in the nearest cluster.

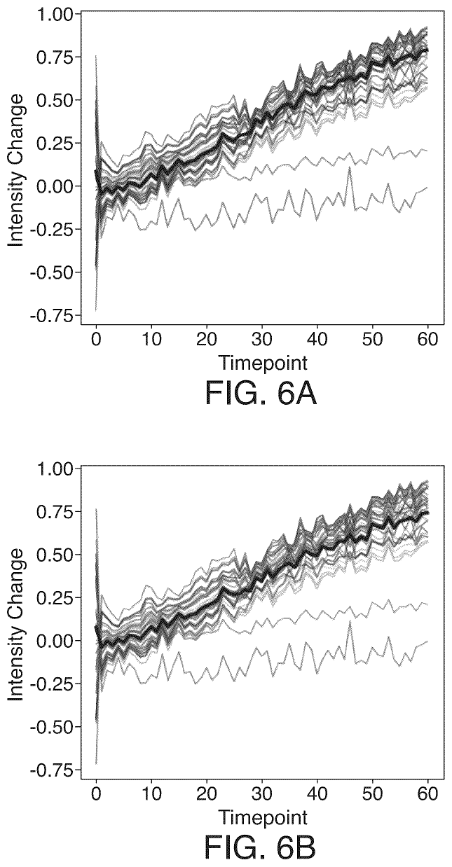

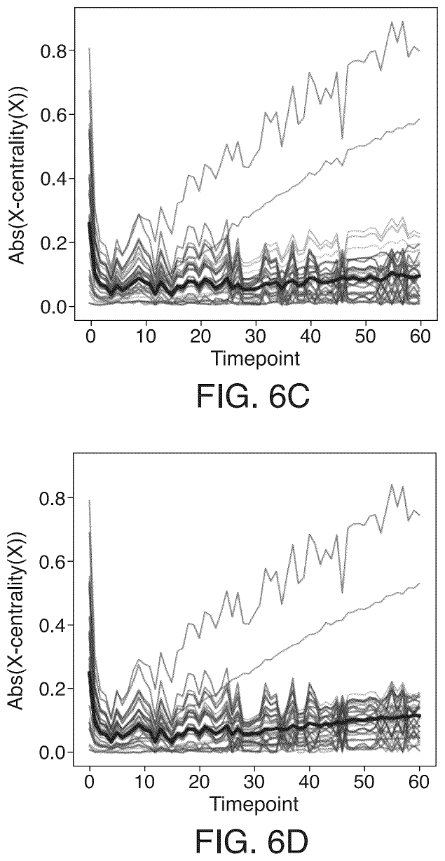

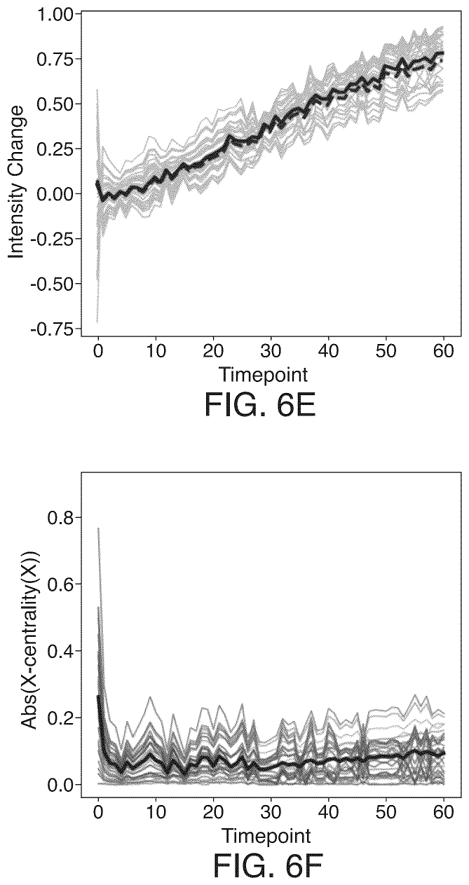

The benefits of performing replicate filtering are illustrated in FIGS. 6A-6F. FIGS. 6A and 6B show replicate intensity (median and mean, respectively) versus time in different wells of the well plate, with failed growth clearly shows as having zero intensity. FIGS. 6C and 6D show the MAD of the intensity, median and mean, respectively, versus time in different wells of the well plate, with failed growth clearly shows as having a substantial deviation away from the wells with observed microbial growth. FIGS. 6E and 6F illustrate the effects of filtering the data, with the black line in FIG. 6E being the mean of the filtered data following k-means clustering and FIG. 6F illustrating the new MAD for the mean filtered data.

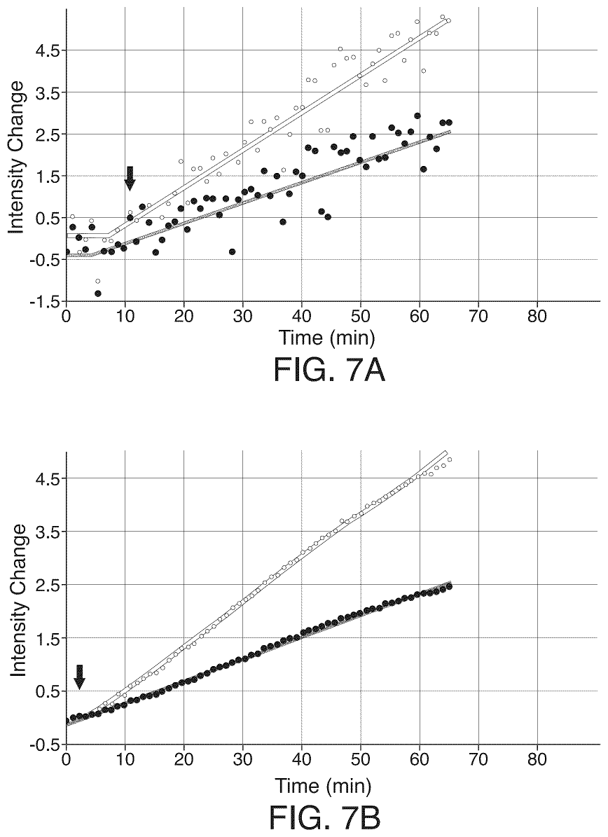

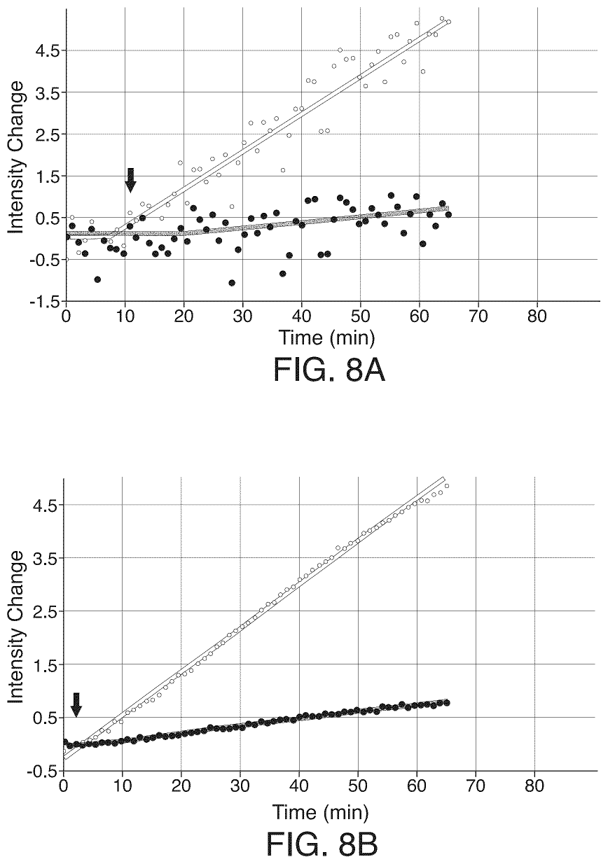

The further effects of filtering out the two minimally growing replicates and the resultant mean measurement, and thus providing a more accurate representation of the aggregate growth and better estimate of the true growth, are illustrated in FIGS. 7A, 7B, 8A and 8B. For example, FIGS. 7A and 7B illustrate an example of a prior growth model in modeling the growth of a microbial species in the presence of 32 μg/mL cefepime using different image analysis schemes. In each of FIGS. 7A and 7B, the dark fit line represents a growth control, i.e., no cefepime, and the light fit line represents growth in the presence of cefepime. As is clear from FIGS. 7A and 7B, compared to a prior image analysis scheme, the image analysis scheme disclosed herein improves the mean absolute error of the prior growth model, from 0.362 (FIG. 7A) to 0.022 (FIG. 7B), respectively. The results illustrated in FIGS. 7A and 7B also suggest that the estimated growth using the image analysis scheme disclosed herein in the prior growth model is slower than predicted by the prior image analysis scheme as applied to the prior growth model.

Similarly, FIGS. 8A-8B illustrate an example of a prior growth model in modeling the growth of a microbial species in the presence of 16 μg/mL ciprofloxacin using different image analysis schemes. In each of FIGS. 8A and 8B, the light fit line represents a growth control, i.e., no ciprofloxacin, and the dark fit line represents growth in the presence of ciprofloxacin. As is clear from FIGS. 8A-8B, the prior image analysis scheme used in the prior model overpredicts growth and has a higher mean absolute error of 0.321 (FIG. 8A) compared to the mean absolute error of 0.122 for the image analysis scheme of the present disclosure (FIG. 8B).

As is clearly seen in FIGS. 7A, 7B, 8A, and 8B, the image analysis scheme disclosed herein increased the confidence to determine growth from sample images collected on shorter timescales. Thus, instead of the average of about 60-70 minutes to claim the observed growth is statistically different than no observed growth under prior image analysis methods, the confidence in the growth determination, using image collection time as a metric, decreased to about 25-35 minutes using the image analysis scheme disclosed herein.

The third step was fitting the de-noised, filtered-mean time series to a novel integrated model based on two standard models in the microbial susceptibility field. As described herein, the Gompertz model is a sigmoidal model of microbial growth that is represented by the following basic equation:

$$= A * exp ( - exp ( b - c x ) )$$

It can be re-written such that the parameters better reflect biological phenomena, where λ is the lag time, and μₘ is the maximum growth rate:

$$y = A * exp \{ - exp [ \frac{{μ}_{m} e}{A} ( λ - t ) + 1 ] \}$$

$$where {μ}_{m} = \frac{A c}{e} and λ = \frac{b - 1}{c}$$

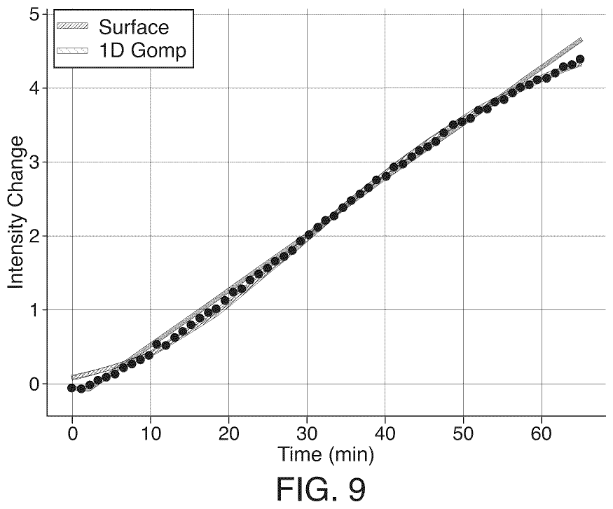

FIG. 9 illustrates a comparison between the modeled growth of a prior growth model, the Gompertz model, and a model according to an embodiment of this disclosure. In each model fit in FIG. 9, the models were fit following the background correction, ICA separation, and replicate filtering steps as described herein in this example. As illustrated in FIG. 9, the Gompertz fit has a lower squared error than the prior model, and the parameters have a better motivated biological interpretation. Another advantage is that the Gompertz model requires three parameters (A, λ, and μₘ) instead of the prior model's four factors (m₀, b₀, m₁, and b₁), and thus the model of this disclosure has less risk of overfitting.

However, overfitting becomes more of a concern when the Gompertz model is fit to each median time series of the several antibiotic concentrations typically of interest in AST. For example, for n concentrations tested, the total number of parameters are 3n, increasing the opportunities for overfitting the data, with the issue of overfitting becoming problematic in models having a 4n parameter space. In additional to risk of overfitting, the Gompertz model may ignore information that can be derived from the changes in growth across concentrations to generate a robust model, which, biologically, should be smooth and thereby statistically interdependent.

Whereas the Gompertz equation models bacterial growth at a given concentration, the Hill equation models the dose response of bacterial growth to antibiotics at a given time. The Hill equation can be represented as:

$$y = A + \frac{{y}_{max}}{1 + {( \frac{k}{{e}^{x}} )}^{n}}$$

The Hill equation is a modified logistic equation, where the inflection point k corresponds to the minimum inhibitory concentration (MIC) of the antibiotic. In practice, the concentration (x) of the antibiotic is exponentiated due to the typical range of antibiotic concentrations (7 to 14-fold dilutions) as the data is fitted to log₂(concentrations). The same caveats regarding overfitting, and overlooking dependency information, apply here as for the Gompertz equation, except across timepoints instead of across concentrations.

To minimize overfitting while taking advantage of dependencies across timepoints and concentrations, the denoised, clean-median time data series was fit to a combined time-concentration-effect model based on the combination of the Gompertz model and the Hill model:

$$y = ( {A}_{A} + \frac{{y}_{{max}_{A}}}{1 + {( \frac{{k}_{A}}{{e}^{x}} )}^{{n}_{A}}} ) * exp \{ - exp [ \frac{( {A}_{μ} + \frac{{y}_{{max}_{μ}}}{1 + {( \frac{{k}_{μ}}{{e}^{x}} )}^{{n}_{μ}}} ) * e}{{A}_{A} + \frac{{y}_{{max}_{A}}}{1 + {( \frac{{k}_{A}}{{e}^{x}} )}^{{n}_{A}}}} ( ( {A}_{λ} + \frac{{L}_{λ}}{1 + {e}^{- {κ}_{λ} ( x - {x}_{o} )}} ) - t ) + 1 ] \}$$

Qualitatively, this equation modeled microbial growth at a given timepoint via the Gompertz curve, but parameterizes the Gompertzian variables (A, λ, μₘ) as functions of three independent Hill functions. As such, the microbial growth data for a given concentration-time domain were simultaneously fit to 12 total parameters, down from the typical 21-42, i.e., 3n, parameters of independent fitting which also ignored important dependencies in the data.

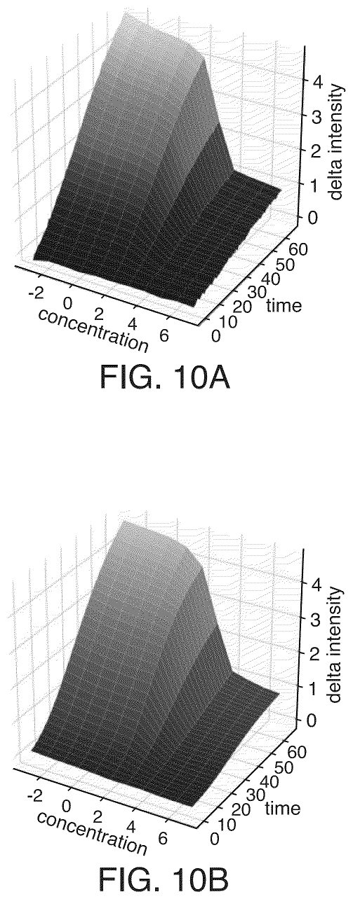

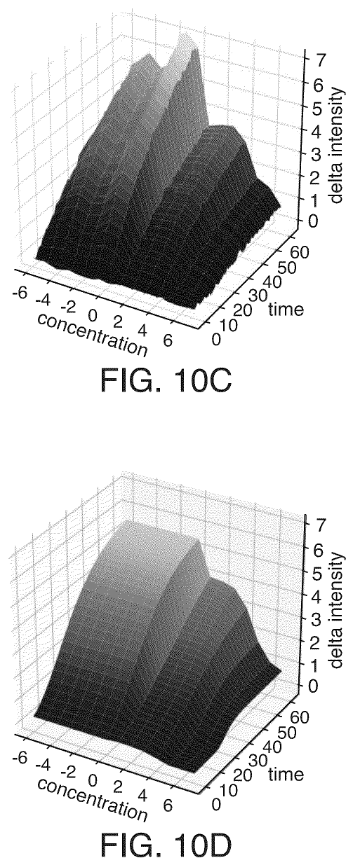

Results of fitting to this hybrid model are illustrated in FIGS. 10A-10D. Note how the concentration-time surface fit did not overfit the data at concentrations where microbial growth is expected to be lower, e.g., log [(concentration)=0]<[log (concentration)=−1] in the raw data, but the model suggests that growth should be equal or less at the higher antibiotic concentration. As illustrated by the red curve in FIG. 9, the time-concentration surface provides a better fit than prior models, but a poorer fit than the 1D Gompertz model, which as described herein predicts the non-biological dependency on concentration as a symptom of overfitting, i.e., its fit is not influenced by the growth patterns of adjacent concentrations.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only. Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

What is claimed is:

1. A system for determining a susceptibility of a microbial species in the presence of an antimicrobial agent, the system comprising:
a) an image collection subsystem constructed and arranged to generate a plurality of images of a microbial sample; and
b) an image analysis subsystem comprising a non-transitory computer-readable medium storing thereon sequences of computer-executable instructions for determining the susceptibility of the microbial species from the image of the microbial sample that, when executed by one or more processors, cause the one or more processors to perform operations comprising:
i) receiving, from the image collection subsystem, one or more of the plurality of images of the microbial sample;
ii) extracting data corresponding to a pixel intensity of one or more regions of the one or more of the plurality of images;
iii) reducing intensity variations in the per pixel intensity of the one or more regions of the one or more of the plurality of images; and
iv) calculating the susceptibility of the microbial species by determining one or both of microbial species replication and microbial species stasis in the presence of the antimicrobial agent from the manipulation of the changing pixel intensities.

2. The system of claim 1, wherein the image collection subsystem comprises a light source, a photosensitive element constructed and arranged to collect light from the light source that has transmitted through the microbial sample, and a memory for storing an image representative of the collected transmitted light from the microbial sample.

3. The system of claim 1, wherein determining the susceptibility of the microbial species comprises one or more of:
a) reducing noise in the pixel intensity of one or more regions of the one or more of the plurality of images;
b) removing statistical outliers from the pixel intensity of the one or more regions of the one or more of the plurality of images; and/or
c) fitting the pixel intensity of the one or more regions of the one or more of the plurality of images to a model representative of a growth dynamic of the microbial species to determine the susceptibility.

4. The system of claim 1, wherein the image analysis subsystem is further configured to display the results of the image analysis to a user.

5. The system of claim 4, wherein the displayed results are used to determine a treatment course for a patient.

6. The system of claim 4, wherein the displayed results are used for epidemiological purposes.

7. The system of claim 1, wherein the microbial species comprises at least one species from the genus Acinetobacter, Escherichia, Klebsiella, Pseudomonas, Enterococcus, Streptococcus, and Staphylococcus.

8. The system of claim 7, wherein the microbial species is selected from A. baumannii, E. coli, K. pneumoniae, P. aeruginosa, and S. aureus.

9. The system of claim 1, wherein the microbial species may be grown for less than or about 12 hours during collection of the plurality of images.

10. The system of claim 1, wherein the microbial species may be grown for less than or about 1.5 hours during collection of the plurality of images.

11. The system of claim 1, wherein the microbial species may be grown for about 1 hour during collection of the plurality of images.

12. The system of claim 1, wherein the microbial sample comprises a well plate having a plurality of wells each separated by at least one surrounding interwell region, the microbial sample including microbial growth in a portion of the plurality of wells.

13. The system of claim 1, wherein the one or more regions of the at least one of the plurality of images correspond to the plurality of wells and the associated at least one surrounding interwell region.

14. The system of claim 1, wherein reducing intensity variations comprises correcting the pixel intensity of the pixels in each of the plurality of wells using the pixel intensities of the associated at least one surrounding interwell region.

15. The system of claim 1, wherein reducing noise comprises performing independent component analysis on the variation reduced pixel intensity data of the pixels in each of the plurality of wells to generate at least one signal corresponding to microbial growth and at least one signal corresponding to growth inhibition from the antimicrobial agent.

16. The system of claim 1, wherein removing statistical outliers comprises performing one or both of a mean absolute deviation calculation and a k-means clustering calculation on the noise reduced pixel intensity data.

17. The system of claim 1, wherein fitting the pixel intensity comprises fitting the outlier reduced pixel intensity data to a growth dynamic model comprising one or more phenomenological models.

18. The system of claim 17, wherein the growth dynamic model comprises a combined multi-dimensional growth dynamic model comprising the Gompertz model and the Hill model.

19. The system of claim 1, wherein the image analysis subsystem is further configured to calculate the minimum inhibitory concentration (MIC) of the antimicrobial agent.

20. A method of determining a susceptibility of a microbial species in the presence of an antimicrobial agent, comprising:
a) acquiring a plurality of images of a microbial sample using an image collection system;
b) sending or transmitting one or more of the plurality of images to an image analysis system comprising a non-transitory computer-readable medium storing thereon sequences of computer-executable instructions for determining the susceptibility of the microbial species from one or more of the plurality of images of the microbial sample by manipulating data corresponding to a pixel intensity of one or more regions of one or more of the plurality of images to a hybrid model representative of a growth dynamic of the microbial species;
c) calculating a minimum inhibitory concentration (MIC) of the antimicrobial agent from the one or more of the plurality of images by determining one or both of microbial species replication and microbial species stasis in the presence of the antimicrobial agent from the determined growth dynamic; and
d) storing or providing the result of part c) to a user.

21. The method of claim 20, wherein step b) further comprises extracting data corresponding to a pixel intensity of one or more regions of the one or more of the plurality of images.

22. The method of claim 20, wherein step b) further comprises reducing intensity variations in the pixel intensity of the one or more regions of the one or more of the plurality of images.

23. The method of claim 20, wherein step b) further comprises reducing noise in the pixel intensity of one or more regions of the one or more of the plurality of images.

24. The method of claim 20, wherein step b) further comprises removing statistical outliers from the pixel intensity of the one or more regions of the one or more of the plurality of images.

25. The method of claim 20, wherein the hybrid model comprises a combined multi-dimensional growth dynamic model comprising the Gompertz model and the Hill model.

26. A non-transitory computer-readable medium storing instructions which, when executed by a computer, cause the computer to perform a method, the method comprising:
a) acquiring a plurality of images of a microbial sample using an image collection system;
b) determining from analysis of one or more of the plurality of images of the microbial sample a growth dynamic including one or both of microbial species replication and microbial species stasis in the presence of an antimicrobial agent; and
c) calculating a minimum inhibitory concentration (MIC) of the antimicrobial agent from the determined microbial growth dynamic in the one or more of the plurality of images of the microbial sample.

27. The non-transitory computer-readable medium of claim 26, wherein the step of determining the growth dynamic comprises determining the growth dynamic using a combined multi-dimensional growth dynamic model comprising the Gompertz model and the Hill model.
